(19) United States
(12) Patent Application Publication
(10) Pub. No.: US 2025/0082557 A1
(43) Pub. Date: Mar. 13, 2025

(54) COMPOSITION FOR ENHANCING SKIN VITALITY CONTAINING A THYMOL ESTER-BASED COMPOUND

(71) Applicant: Amorepacific Corporation, Seoul (KR)

(72) Inventors: Hyunjung CHOI, Yongin-si (KR); Dongryeol Ryu, Suwon-si (KR); Eun Ju Jin, Suwon-si (KR); Shibo Wei, Gwangju (KR)

(73) Assignee: Amorepacific Corporation, Seoul (KR)

(21) Appl. No.: 18/829,496

(22) Filed: Sep. 10, 2024

(30) Foreign Application Priority Data
Sep. 13, 2023 (KR) 10-2023-0121927

(51) Int. Cl. A61K 8/37; A61K 31/25; A61Q 19/00

(52) U.S. Cl. CPC A61K 8/37; A61K 31/25; A61Q 19/00

(57) ABSTRACT

A composition capable of improving skin vitality reduced by stress is disclosed. The composition may include a thymol ester-based compound as an active ingredient, thereby promoting ATP production through mitochondrial activity and further restoring the vitality of skin cells reduced by stress. The composition may be formulated as cosmetic, pharmaceutical, dietary supplements, or food products.

### CROSS REFERENCE TO RELATED APPLICATION

The present application claims priority to Korean Patent Application No. 10-2023-0121927 filed on Sep. 13, 2023, the entire contents of which are incorporated herein by reference.

### BACKGROUND OF THE INVENTION

### Field of the Invention

Herein is disclosed a composition for enhancing skin vitality, including a thymol ester-based compound, wherein the composition is capable of improving skin vitality that is reduced due to stress.

### Description of the Related Art

Factors that cause skin stress include ultraviolet rays, harmful environments, excessive use of cosmetics, psychological stress, etc.

When the skin is stressed due to these various factors, the skin becomes sensitive and the skin regeneration ability decreases, which reduces skin elasticity and makes the skin color dull. In addition, there is a problem that skin vitality decreases and skin troubles occur due to harmful environments or psychological stress.

However, research on the specific mechanism of skin vitality decreased due to stress or method for improving the skin vitality is insufficient. Therefore, research on a method for improving skin vitality from the cellular stage is needed.

### SUMMARY OF THE INVENTION

### Technical Problem

In one aspect of the present disclosure, it is to provide a composition capable of recovering skin vitality reduced by stress through mitochondrial activation.

### Technical Solution

In one aspect, the present disclosure provides a composition for enhancing skin vitality, including 3,4,5-trimethoxycinnamate thymol ester or a stereoisomer, salt, hydrate, or solvate thereof as an active ingredient.

### Advantageous Effects

In one aspect, the composition according to the present disclosure can improve skin vitality through mitochondrial activation.

In another aspect, the composition according to the present disclosure can improve skin vitality by promoting ATP production.

### BRIEF DESCRIPTION OF THE DRAWINGS

FIG. 1 shows a procedure of an oxygen consumption rate (OCR) test according to an embodiment of the present disclosure.

FIG. 2 is a graph illustrating mitochondrial activity, i.e., oxygen consumption rate (OCR), according to an embodiment of the present disclosure.

FIGS. 3 to 5 are graphs showing expression results of mitophagy marker genes (VDAC, Parkin, PINK) according to an embodiment of the present disclosure.

### DETAILED DESCRIPTION OF THE INVENTION

Hereinafter, the present disclosure will be described in more detail by way of the following examples. However, the following examples are provided only for illustrative purposes to aid understanding of the present disclosure, and the scope and range of the present disclosure are not limited thereto.

In exemplary embodiments of the present disclosure, there is provided a composition for enhancing skin vitality, including 3,4,5-trimethoxycinnamate thymol ester or a stereoisomer, salt, hydrate, or solvate thereof as an active ingredient.

In another exemplary embodiment of the present disclosure, there is provided a method for improving skin vitality, including administering an effective amount of a composition for enhancing skin vitality, comprising 3,4,5-trimethoxycinnamate thymol ester or a stereoisomer, salt, hydrate, or solvate thereof as an active ingredient.

In still another exemplary embodiment of the present disclosure, there is provided a use of 3,4,5-trimethoxycinnamate thymol ester or a stereoisomer, salt, hydrate, or solvate thereof for preparing a composition for enhancing skin vitality.

In yet another exemplary embodiment of the present disclosure, there is provided a non-therapeutic use of 3,4,5-trimethoxycinnamate thymol ester or a stereoisomer, salt, hydrate, or solvate thereof for improving skin vitality.

In yet another exemplary embodiment of the present disclosure, there is provided 3,4,5-trimethoxycinnamate thymol ester or a stereoisomer, salt, hydrate, or solvate thereof for improving skin vitality.

In one embodiment, the 3,4,5-trimethoxycinnamate thymol ester is (5-methyl-2-propan-2-ylphenyl) (E)-3-(3,4,5-trimethoxyphenyl) prop-2-enoate, which may be represented by Chemical Formula 1 below:

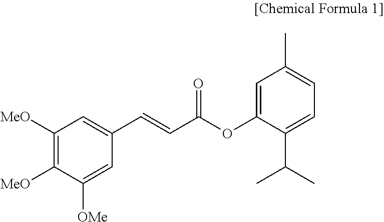 [SMILES: COc1cc(/C=C/C(=O)Oc2cc(C)ccc2C(C)C)cc(OC)c1OC]

As used herein, the term “isomer” includes in particular optical isomers (e.g., essentially pure enantiomers, essentially pure diastereomers or mixtures thereof), as well as conformation isomers (i.e., isomers which differ only in the angles of one or more chemical bonds), position isomers (in particular, tautomers) or geometric isomers (e.g., cis-trans isomers).

As used herein, the term “essentially pure”, for example, when used in connection with an enantiomer or diastereoisomer, means that a specific compound, for example an enantiomer or diastereoisomer, is present in an amount of at least about 90%, preferably at least about 95%, more preferably at least about 97% or at least about 98%, even more preferably at least about 99%, and even more preferably at least about 99.5% (w/w).

As used herein, the term “pharmaceutically acceptable” means that it can be or has been approved by the government or equivalent regulatory body as being suitable for use in animals, and more particularly, in humans, by avoiding significant toxic effects when used in conventional medicinal dosages, or is listed in a pharmacopoeia or recognized in other general pharmacopoeias.

As used herein, the term “pharmaceutically acceptable salt” means a salt according to one aspect of the present disclosure that is pharmaceutically acceptable and has the desired pharmacological activity of the parent compound. The salt may include (1) an acid addition salt formed of inorganic acid such as hydrochloric acid, hydrobromic acid, sulfuric acid, nitric acid, phosphoric acid, etc.; or formed of organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2,2,2]-oct-2-ene-1-carboxylic acid, glucoheptonic acid, 3-phenylpropionic acid, trimethylacetic acid, tert-butylacetic acid, lauryl sulfate, gluconic acid, glutamic acid, hydroxynaphthoic acid, salicylic acid, stearic acid, muconic acid; or (2) a salt formed when an acidic proton present in the parent compound is substituted.

As used herein, the term “hydrate” refers to a compound to which water is bound, and is a broad concept that includes an inclusion compound with no chemical bonding force between water and the compound.

As used herein, the term “solvate” refers to a high-order compound formed between a molecule or ion of a solute and a molecule or ion of a solvent.

In one embodiment, the skin vitality enhancement may be at least one of mitochondrial activity enhancement, mitophagy activity enhancement, ATP production enhancement, and stress hormone activity inhibition.

In one embodiment, the stress hormone may be hydrocortisone.

In one embodiment, the 3,4,5-trimethoxycinnamate thymol ester or a stereoisomer, salt, hydrate, or solvate thereof may be administered at a dosage of 1 μg/kg to 1000 mg/kg.

In one embodiment, the concentration of the 3,4,5-trimethoxycinnamate thymol ester or a stereoisomer, salt, hydrate or solvate thereof may be from 0.0005 to 1 wt % based on the total weight of the composition.

For example, the concentration of the 3,4,5-trimethoxycinnamate thymol ester or a stereoisomer, salt, hydrate or solvate thereof may be 0.0005 wt % or more, 0.001 wt % or more, 0.005 wt % or more, 0.01 wt % or more, 0.1 wt % or more, or 0.5 wt % or more, and 1 wt % or less, 0.5 wt % or less, 0.1 wt % or less, 0.01 wt % or less, or 0.005 wt % or less, or 0.001 wt % or less based on the total weight of the composition.

For example, the concentration of the 3,4,5-trimethoxycinnamate thymol ester or a stereoisomer, salt, hydrate or solvate thereof may be 0.01 to 0.5 wt % based on the total weight of the composition.

In one embodiment, the composition may be applied to a subject whose skin vitality is reduced by stress.

In one embodiment, the composition may be applied to a subject whose mitochondrial activity is decreased. For example, the composition may be applied to a subject with decreased mitochondrial activity in skin cells due to stress, thereby improving the vitality of skin or skin cells.

In relation to the mitochondrial activity, mitophagy is an intracellular degradation mechanism that removes damaged or unnecessary mitochondria, and is important for regulating mitochondrial function in various cells and maintaining tissue function.

In one embodiment, the composition may be applied to a subject whose mitophagy activity is decreased. For example, the composition may be applied to a subject with decreased mitophagy activity in skin cells due to stress, thereby improving the vitality of skin or skin cells.

In one embodiment, the composition may be applied to a subject whose ATP production is decreased. For example, the composition may be applied to a subject with decreased ATP production in skin cells due to stress, thereby improving the vitality of skin or skin cells.

In one embodiment, the composition may be a composition for skin external application. The composition for skin external application is a generic term that may include anything that is applied externally to the skin, and may include various types of cosmetics and pharmaceuticals.

In one embodiment, the composition may be a food composition. The food composition may be a liquid or solid formulation, and may be a tablet, a capsule, a soft capsule, a pills, a granule, a beverage (drinking agent), a diet bar, a chocolate, a caramel formulation, or a confectionery formulation, but the formulation is not particularly limited. In addition to the above active ingredients, the food composition according to the present disclosure may contain excipients, sugars, flavors, pigments, fats, proteins, etc., as needed.

In one embodiment, the composition may be a cosmetic composition.

The external form of the cosmetic composition contains a cosmetically or dermatologically acceptable medium or base. It may be provided in any formulation suitable for topical application, for example, in the form of a solution, a gel, a solid, a pasty anhydrous product, an emulsion obtained by dispersing an oil phase in an aqueous phase, a suspension, a microemulsion, a microcapsule, a microgranule, or ionic (liposome) and nonionic vesicular dispersion, or in the form of a cream, a skin, a lotion, a powder, an ointment, a spray, or a concealer stick. These compositions can be prepared according to conventional methods in the art. The composition according to the present disclosure may also be used in the form of a foam or in the form of an aerosol composition further containing a compressed propellant.

The cosmetic composition according to one embodiment of the present disclosure is not particularly limited in its formulation, and may be formulated into cosmetics such as a softening toner, an astringent toner, a nourishing toner, a nourishing cream, a massage cream, an essence, an eye cream, an eye essence, a cleansing cream, a cleansing foam, a cleansing water, a pack, a powder, a body lotion, a body cream, a body oil, and a body essence.

When the formulation of the cosmetic composition of the present disclosure is a paste, a cream, or a gel, animal fiber, plant fiber, wax, paraffin, starch, tragacanth, a cellulose derivative, polyethylene glycol, silicone, bentonite, silica, talc, or zinc oxide may be used as a carrier component.

When the formulation of the cosmetic composition of the present disclosure is a powder or spray, lactose, talc, silica, aluminum hydroxide, calcium silicate or polyamide powder may be used as a carrier component, and in particular, in the case of a spray, a propellant such as chlorofluorohydrocarbon, propane/butane or dimethyl ether may be additionally included.

When the formulation of the cosmetic composition of the present disclosure is a solution or an emulsion, a solvent, a solvating agent or an emulsifying agent is used as a carrier component, and the examples include water, ethanol, isopropanol, ethyl carbonate, ethyl acetate, benzyl alcohol, benzyl benzoate, propylene glycol, 1,3-butyl glycol oil, glycerol aliphatic ester, polyethylene glycol or a fatty acid ester of sorbitan.

When the formulation of the cosmetic composition of the present disclosure is a suspension, a liquid diluent such as water, ethanol or propylene glycol, a suspending agent such as ethoxylated isostearyl alcohol, polyoxyethylene sorbitol ester and polyoxyethylene sorbitan ester, microcrystalline cellulose, aluminum metahydroxide, bentonite, agar, tragacanth, or the like may be used as a carrier component.

When the formulation of the cosmetic composition of the present disclosure is a surfactant-containing cleansing, aliphatic alcohol sulfate, aliphatic alcohol ether sulfate, sulfosuccinic acid monoester, isethionate, imidazolinium derivative, methyl taurate, sarcosinate, fatty acid amide ether sulfate, alkylamidobetaine, fatty alcohol, fatty acid glyceride, fatty acid diethanolamide, vegetable oil, linoline derivative, ethoxylated glycerol fatty acid ester, etc. may be used as a carrier ingredient.

The cosmetic composition of the present disclosure may further include a functional additive and a component included in a general cosmetic composition in addition to the above-described active ingredients. The functional additive may include a component selected from the group consisting of water-soluble vitamins, oil-soluble vitamins, polymer peptides, polymer polysaccharides, sphingo lipids, and seaweed extract.

In the cosmetic composition of the present disclosure, in addition to the above functional additive, a component included in a general cosmetic composition may be blended as necessary. In addition, other blending components, such as oil and fat ingredients, moisturizers, emollients, surfactants, organic and inorganic pigments, organic powders, ultraviolet absorbers, preservatives, disinfectants, antioxidants, plant extracts, pH adjusters, alcohols, pigments, fragrances, blood circulation promoters, cooling agents, anhydrotics, purified water, etc., may be additionally included.

In one embodiment, the composition may be a pharmaceutical composition.

In one embodiment, the pharmaceutical composition may be for treating a subject whose skin vitality is reduced by stress.

In one embodiment, the pharmaceutical composition may be for treating a subject whose mitochondrial activity is reduced.

In one embodiment, the pharmaceutical composition may be for treating a subject whose mitophagy activity is reduced.

In one embodiment, the pharmaceutical composition may be for treating a subject whose ATP production is reduced.

In one embodiment, the composition may be for preventing or improving skin aging.

In one embodiment, the composition may be for preventing, improving, or treating a disease related to skin mitochondrial dysfunction.

For example, the disease related to the skin mitochondrial dysfunction or disorder may include at least one selected from the group consisting of psoriasis, atopic dermatitis (eczema), porphyria cutanea tarda, acne, and rosacea.

As used herein, the term “prevention” means any action of suppressing a disease or delaying the onset of a disease by administering the pharmaceutical composition according to the present disclosure.

As used herein, the term “improvement” refers to any action that at least reduces a parameter related to the condition being treated, for example, the degree of symptoms.

As used herein, the term “treatment” refers to any action that ameliorates or beneficially changes symptoms of a disease by administering the pharmaceutical composition according to the present disclosure.

The pharmaceutical composition may additionally contain pharmaceutical adjuvants such as preservatives, stabilizers, hydrating agents or emulsifying agents, salts for osmotic pressure control, and/or buffers, and other therapeutically useful substances, and may be formulated into various oral or parenteral dosage forms according to conventional methods.

The oral dosage forms include, for example, tablets, pills, hard and soft capsules, solutions, suspensions, emulsions, syrups, dusts, powders, fine granules, granules, pellets, etc., and these formulations may contain, in addition to the active ingredient, a surfactant, a diluent (e.g., lactose, dextrose, sucrose, mannitol, sorbitol, cellulose, and glycine), and a lubricant (e.g., silica, talc, stearic acid and its magnesium or calcium salts, and polyethylene glycol). The tablet may also contain a binder such as magnesium aluminum silicate, starch paste, gelatin, tragacanth, methylcellulose, sodium carboxymethylcellulose, and polyvinylpyrrolidine, and may optionally contain pharmaceutical additives such as a disintegrant, an absorbent, a coloring agent, a flavoring agent, and a sweetener, such as starch, agar, alginic acid or a sodium salt thereof. The tablet may be manufactured by a conventional mixing, granulating, or coating method. In addition, the parenteral dosage form may be a transdermal dosage formulation, for example, a formulation such as an injection, a drop, an ointment, a lotion, a gel, a cream, a spray, a suspension, an emulsion, a suppository, a patch, etc., but is not limited thereto.

The pharmaceutical composition according to the present disclosure may be administered parenterally, rectally, topically, transdermally, subcutaneously, etc. The pharmaceutical composition according to the present disclosure may be administered topically, for example, to the scalp.

The determination of the dosage of the above-mentioned active ingredient is within the level of those skilled in the art, and the daily administration dose of the drug varies depending on various factors such as the degree of progression, onset time, age, health condition, complications, etc. of the subject to be administered, but based on adults, may generally be 1 μg/kg to 1000 mg/kg of the composition, for example, 0.1 mg/Kg to 20 mg/Kg, 0.5 mg/Kg to 20 mg/Kg, or 1 mg/kg to 20 mg/kg, preferably 5 mg/kg to 10 mg/kg, which may be divided and administered 1 to 3 times a day, and the dosage does not limit the scope of the present disclosure in any way.

Hereinafter, the present disclosure will be described in more detail by way of the following examples. However, the following examples are provided only for illustrative purposes to aid understanding of the present disclosure, and the scope and range of the present disclosure are not limited thereto.

### EXAMPLES

Preparation of 3,4,5-trimethoxycinnamate thymol ester Thymol trimethoxycinnamate thymol ester with CAS number 504394-57-4 (MELASOLV™) was obtained from COSMANN Co., Ltd. (Hwaseong-si, Gyeonggi-do, Korea).

### Test Example

A skin fibroblast was treated with stress hormone hydrocortisone to confirm whether mitochondrial activity is reduced, and an effect of improving mitochondrial activity by MELASOLV™ (3,4,5-trimethoxycinnamate thymol ester) was confirmed. Also, it was confirmed whether the increase in mitochondrial activity was due to the activation of mitophagy occurring in mitochondria.

### Test Example 1—Mitochondrial Activity

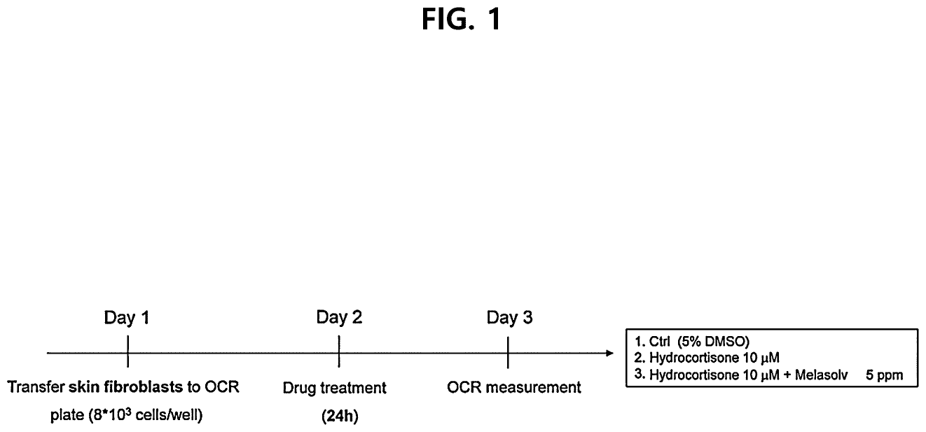

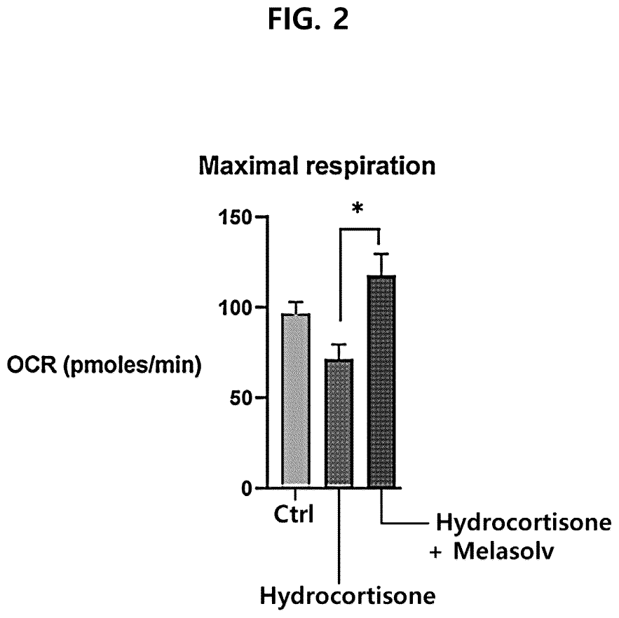

As shown in FIG. 1, skin fibroblasts were transferred to an OCR plate (8*10³ cells/well) and treated with stress hormone hydrocortisone (10 uM, 24 hours) and MELASOLV™ 5 ppm for 24 hours, respectively, to observe the mitochondrial activity (ATP energy production efficiency), i.e., the maximal oxygen consumption rate (OCR). Specifically, the Seahorse XF Pro analyzer equipment from Agilent was used, and the mitochondrial activity (ATP energy production efficiency), i.e., the maximal oxygen consumption rate (OCR), was observed according to the standard protocol provided with the Seahorse XF cell mitochondrial stress measurement kit provided by the company (FIG. 2).

Referring to FIG. 2, the results of observing the maximal respiration showed a statistically significantly higher value in the case of treatment with MELASOLV™ (5 ppm, 24 hours) compared to the case of treatment with only hydrocortisone, which could confirm that the value increased higher even compared to the case of the control group which is not treated with anything.

In conclusion, compared to the control group (Ctrl), the cell respiration rate (OCR) was reduced under the condition treated with hydrocortisone (stress-induced condition), and it can be confirmed that it was recovered when treated with MELASOLV™. In conclusion, the function of mitochondria inhibited by stress was recovered by MELASOLV™ treatment.

### Test Example 2-Expression of Mitophagy Marker Genes (VDAC, Parkin, PINK)

Fibroblasts were seeded in 10 cm² cell culture dishes at a concentration of 3×10⁵ cells/dish and cultured in 10 ml of minimum essential medium eagle (MEM) medium containing 10% fetal bovine serum (FBS) and 1% penicillin streptomycin. After 24 hours, the cells reached approximately 70%, and the cells were treated with (1) DMSO (control), (2) hydrocortisone (10 μM) alone, or (3) hydrocortisone (10 μM) and MELASOLV™ (5.5 μg/ml) for 24 hours.

RNA was extracted with easy-BLUE (INTRON) reagent. cDNA samples were generated from 0.5 μg of RNA using PrimeScript RT reagent Kit. The generated cDNA was amplified using ACCUPOWER™ PCR Premix. The PCR products were further analyzed using PCR Thermal Cycler Dice.

It was confirmed through a decrease in the expression of mitophagy marker genes (VDAC, Parkin, PINK) whether it contributes to the activation of mitophagy which removes unnecessary elements from mitochondria.

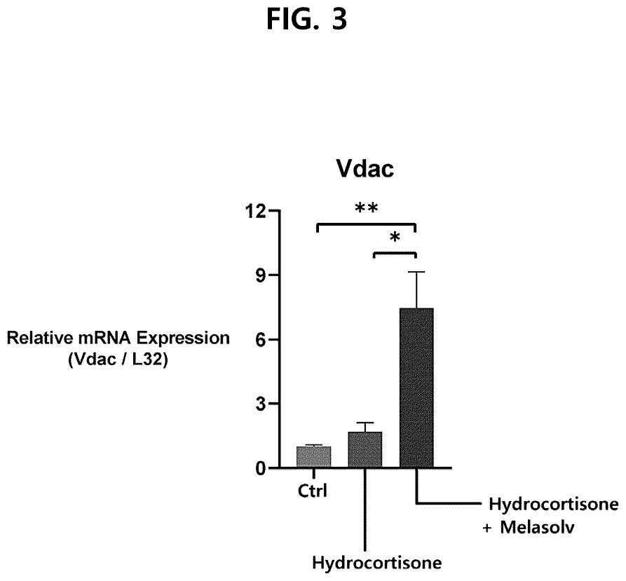

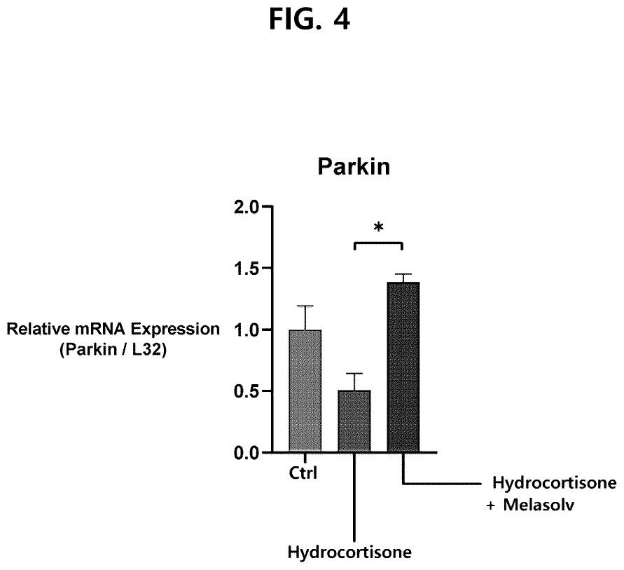

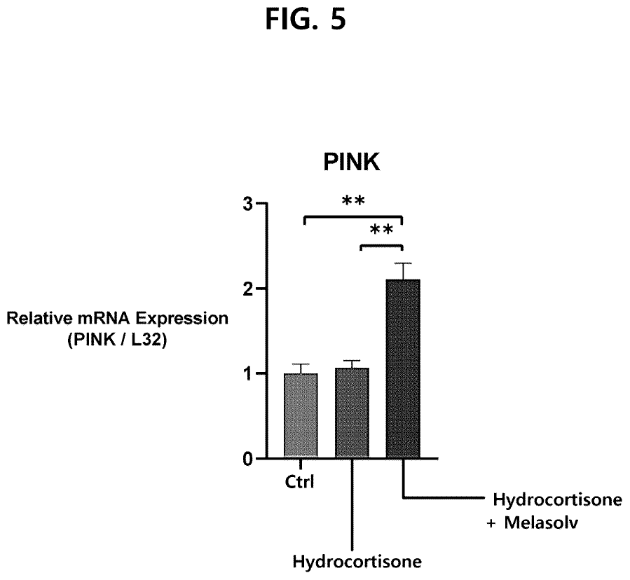

Referring to FIGS. 3 to 5, when treated with MELASOLV™, it was confirmed that the expression of mitophagy genes increased and was caused by mitophagy activation.

According to the present disclosure, a decrease in skin vitality (decrease in ATP production) due to stress hormones can be recovered by treatment with 3,4,5-trimethoxycinnamate thymol ester, which can be expected to be significantly recovered compared to the control group and even increase to a level higher than the normal state.

In addition, 3,4,5-trimethoxycinnamate thymol ester can be presented as a material that promotes skin energy production through the activation of mitophagy, a type of autophagy that occurs in mitochondria, and by including the same, a composition that recovers a decrease in skin vitality and skin life caused by stress can be provided.

What is claimed is:

1. A method for enhancing skin vitality, comprising administering an effective amount of a composition comprising 3,4,5-trimethoxycinnamate thymol ester or a stereoisomer, salt, hydrate, or solvate thereof as an active ingredient to a subject in need thereof.

2. The method according to claim 1, wherein the skin vitality enhancement comprises at least one of mitochondrial activity enhancement, mitophagy activity enhancement, ATP production enhancement, and stress hormone activity inhibition.

3. The method according to claim 2, wherein the stress hormone is hydrocortisone.

4. The method according to claim 1, wherein the 3,4,5-trimethoxycinnamate thymol ester or the stereoisomer, salt, hydrate, or solvate thereof is administered at a dosage of 1 μg/kg to 1000 mg/kg.

5. The method according to claim 1, wherein the concentration of the 3,4,5-trimethoxycinnamate thymol ester or the stereoisomer, salt, hydrate, or solvate thereof is 0.0005 to 1 wt % based on the total weight of the composition.

6. The method according to claim 1, wherein the composition is applied to a subject whose skin vitality is reduced by stress.

7. The method according to claim 1, wherein the composition is applied to a subject whose mitochondrial activity is decreased.

8. The method according to claim 1, wherein the composition is applied to a subject whose mitophagy activity is decreased.

9. The method according to claim 1, wherein the composition is applied to a subject whose ATP production is decreased.

10. The method according to claim 1, wherein the composition is a composition for skin external application.

11. The method according to claim 1, wherein the composition is a food composition.

12. The method according to claim 1, wherein the composition is a cosmetic composition.

13. The method according to claim 1, wherein the composition is a pharmaceutical composition.
